(19) United States
(12) Patent Application Publication
(10) Pub. No.: US 2014/0107408 A1
(43) Pub. Date: Apr. 17, 2014

(54) DEVICE FOR CORRECTING SUNKEN CHEEKS

(71) Applicant: Sylvia Tatevosian Rostami, Tarzana, CA (US)

(72) Inventor: Sylvia Tatevosian Rostami, Tarzana, CA (US)

(21) Appl. No.: 14/123,308

(22) PCT Filed: Jun. 6, 2012
(86) PCT No.: PCT/US2012/040968

§ 371 Date: Dec. 2, 2013

Related U.S. Application Data
(60) Provisional application No. 61/520,082, filed on Jun. 6, 2011.

(51) Int. Cl. A61F 5/01

(52) U.S. Cl. CPC A61F 5/01
USPC 600/37

(57) ABSTRACT

This invention relates to a cheek plumping apparatus which is comprised of one or multiple cheek pads and the means of attaching the cheek pads to the outer face of an individual's hind teeth. This invention thus causes the individual's cheeks to be pushed outwards and plumped, thereby giving them a more youthful appearance. The invention includes both removable and permanent means of attaching the cheek pads.

### TECHNICAL FIELD

This invention relates to an apparatus wherein one or multiple cheek pads, combined with a means of attachment of the cheek pads (either removable or permanent), are attached to the outer face of an individual's hind teeth, causing the individual's cheeks to be pushed outwards, plumping the individual's cheeks, and giving them a more youthful appearance.

### BACKGROUND ART

Aging brings about many changes to people's faces. One significant change that occurs to one's face is how our cheeks gradually become hollowed, sunken and deflated looking. A significant contributor to the youthful look we had as children and young adults were full, round cheeks. As we age, our skin and malar fat pads become thinner, resulting in sunken cheeks.

Several solutions have been developed in the past to try to address this problem. Cheek implants are silicone structures that are attached to the cheek bones to lift up the cheeks. However, even sub-malar implants, which are placed on the lower portion of the cheek bone, do not significantly address the sunken look in the mouth (buccalis) region of the cheeks.

Various dermal fillers are also used to try to fill in this sunken cheek region. For instance, Restylane or Juvederm can be injected into the cheek area, but these are temporary, and are resorbed within months. Autologous fat injection can be a more permanent option, but this is very costly, and results can vary from retention, partial retention, to complete resorbtion. In addition, many people have concerns about having such procedures done because they are not willing to go “under the knife”, or fear possible complications from such implants and injections.

A few devices have also been developed that are placed inside the mouth to fill in and smooth wrinkles of the face. For example, U.S. Pat. No. 2003/0023313 A1 (Byers) describes cheek pads for diminishing the appearance of depressions of the cheeks. However, this patent provides only for the cheek pads, and not for the means of attachment of the cheek pads, having the pressure of the cheeks and the custom impressioning of the cheek pads on the hind teeth be the primary means of preventing the cheek pads from dislodging. This patent does mention once using denture adhesive sometimes to secure the cheek pad in place, after custom impressioning, but as has been demonstrated with Charles Pincus' veneers, which fell off often due to being attached to the teeth using denture adhesive, denture adhesive is not an effective means of attachment to hold cheek pads securely in place, and prevent them from becoming dislodged.

U.S. Pat. No. 1,0539,433 (Bruce) describes a cosmetic prostheses which sits on an individual's gums to push out and diminish wrinkles around the lips and corners of the mouth. However, this invention's plumping component sits only on the gums, not on the teeth, and relates to diminishing wrinkles around the lips and corners of the mouth, without addressing the means for plumping the cheek regions of the face.

### SUMMARY OF INVENTION

Thus to make a cheek plumping apparatus viable, it is necessary for the cheek pads to cover not only the hind teeth's gums, but also at least some of the teeth as well, in order for the cheeks to be filled completely and naturally. In addition, it is necessary to have a secure means of attachment being part of the cheek plumping apparatus, in order to avoid having the cheek pads dislodge and move about in the mouth.

This invention provides a solution to the above stated problems. In this invention, one or multiple cheek pads, combined with a means of attachment (either removable or permanent) of the cheek pads, are attached to the outer face of an individual's hind teeth, causing the individual's cheeks to be pushed outwards, thereby plumping the individual's cheeks and giving them a more youthful appearance.

### BRIEF DESCRIPTION OF THE DRAWING

This invention can be better understood via the following drawings and their descriptions:

FIG. 1 illustrates a cheek plumping apparatus comprised of one cheek pad, and two molar bands as means of attachment.

FIG. 2 illustrates a cheek plumping apparatus comprised of four cheek pads, four molar bands with headgear tubes, and two holding arches, as seen from the top, side, and front view of the mouth.

FIG. 3 illustrates a maxilla and a mandible cheek pad from the side view.

FIG. 4 illustrates placing a cheek pad in each quadrant.

FIG. 5 illustrates placing a cheek pad in each quadrant of the maxilla.

FIG. 6 illustrates a vacuum formed retainer with two cheek pads bonded onto it.

FIG. 7 illustrates a vacuum formed retainer with two cheek pads that are made to look like an extension of the individual's teeth bonded onto it.

FIG. 8 illustrates pre-manufactured cheek pads in three sizes.

FIG. 9 illustrates a Hawley retainer as being the removable dental appliance like structure which serves as the means of attachment of two cheek pads.

FIG. 10 illustrates a partial denture (without denture teeth) as being the removable dental appliance like structures which serves as the means of attachment of two teeth like cheek pads.

FIG. 11 illustrates a Hawley retainer as being the functional removable dental appliance with two cheek pads.

FIG. 12 illustrates a full denture with removable means of attaching two teeth and gum like cheek pads.

FIG. 13 illustrates a full denture with two teeth and gum like cheek pads permanently incorporated into it.

### GENERAL DISCLOSURE

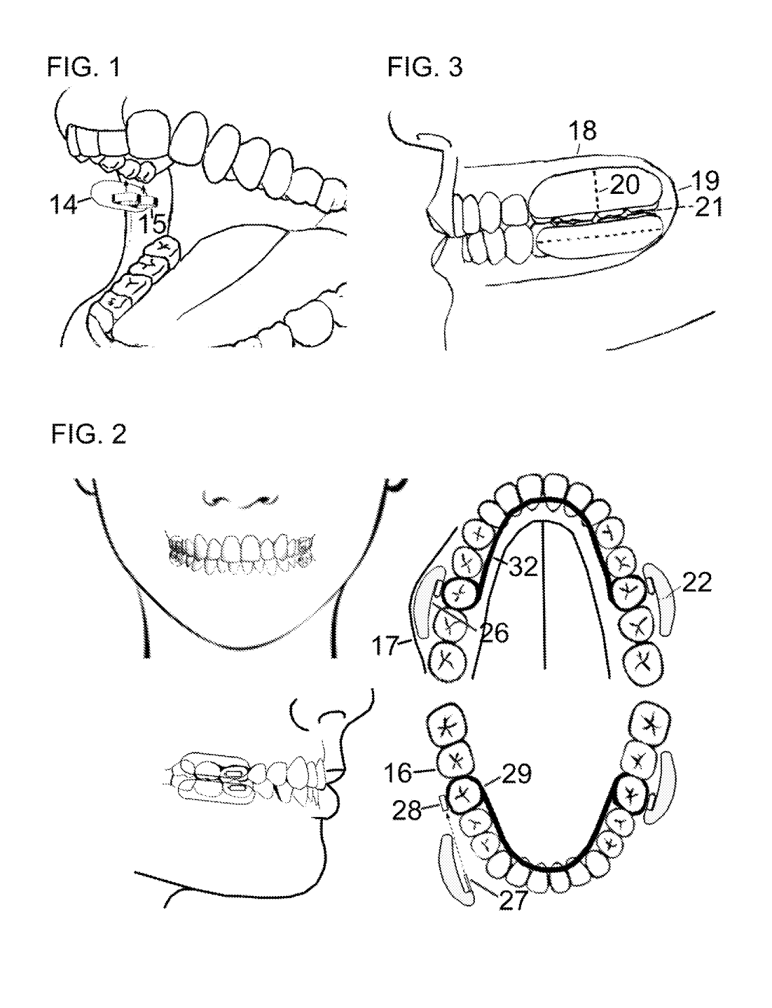

In this invention, one or multiple cheek pads (FIG. 1, 14), combined with a biocompatible means of attachment (FIG. 1, 15) of the cheek pads (either removable or permanent), are attached to the outer face of an individual's hind teeth (FIG. 2, 16), causing the individual's cheeks to be pushed outwards (FIG. 2, 17), thereby plumping the individual's cheeks and giving them a more youthful appearance. Thus, a cheek plumping apparatus is defined as at least one cheek pad plus the means of attaching the cheek pad to at least one of the individual's hind teeth. For the purpose of this invention, teeth is defined as both natural and simulation teeth. And hind teeth are defined as at least one of the individual's most posterior teeth, but can include everything from the wisdom teeth to the first bicuspid.

In this invention, a cheek pad is a thick piece of biocompatible material or composite of biocompatible materials that are safe to use in a human mouth. It fits comfortably inside an individual's mouth in the hind quadrant's space between the inner face of the cheek (FIG. 2, 17) and the outer face of the hind teeth (FIG. 2, 16). In order to not irritate any of the tissues around it, the cheek pad's size cannot be so large as to push on and irritate the mucobuccal fold (FIG. 3, 18) or the anterior folds in the cheek's cavity (FIG. 3, 19). In addition, the height of the cheek pad (FIG. 3, 20) must be such that it does not interfere with the movement of one's mouth during talking, etc. or to obstruct the individual from being able to close his/her mouth and jaws completely. So for instance, if a cheek pad is attached to both the maxilla and mandible hind teeth, and if both maxilla and mandible cheek pads were to extend past the molar's biting surface (FIG. 3, 21), then they would prevent the individual from being able to bite and completely close his/her mouth.

Another concern which relates to the height of the cheek pads (FIG. 3, 20) is whether the pad will be located over the hind teeth, the hind gums, or both. Each pad's height and location can be such that it sits over at least part of the hind teeth of the individual, not the gums. Each pad's height and location can be such that it sits over at least part of the hind gums of the individual, not the teeth. Or, each pad's height and location can be such that it covers at least part of the hind teeth as well as at least part of the hind gums. It is preferable that the height of the cheek pad be the highest that will comfortably and safely fit in the individual's mouth, while meeting the requirements put forth in the preceding paragraphs. This technique will optimize the cheeks smoothly draping over the cheek pads, without risking any unnatural poked out regions or sunken in regions.

With respect to thickness (FIG. 2, 22), depending on the age or needs of the individual, a thicker or thinner cheek pad will achieve the most natural and desirable results. Too thick of a cheek pad could cause the cheek to swell out unnaturally, and too thin of a cheek pad would not adequately fill in the cheek. Thus, in most cases, depending on each individual's needs, the cheek pad's thickness will range anywhere from half a centimeter to two centimeters.

The cheek pad's shape can vary, so long as it meets the requirements stated in the former paragraphs. Some shapes for the cheek pad include, but are not limited to being rectangular, oval, or irregular. It can be convex on its exterior side, or primarily flat.

The cheek pad can be made of many different materials, so long as it is a biocompatible material or composite of biocompatible materials that are safe to use in a human mouth. The cheek pad's material must be durable, such that it will not tear or break apart while in use, and it must be solid at or below body temperature. Also, the cheek pad's material can be either hard or soft and flexible, but its surface (or its coating) must be such that it will not irritate or otherwise disturb the mouth's tissues or compromise the health of the individual wearing it when worn for hours at a time. Thus, an unlimited number and combination of materials can be used to make the cheek pad. Some such materials that the cheek pads can be made from include, but are not limited to metal, silicon, acrylic, plastic and other polymer derived materials, hydrogel, ceramic, glass, or any composite of such materials.

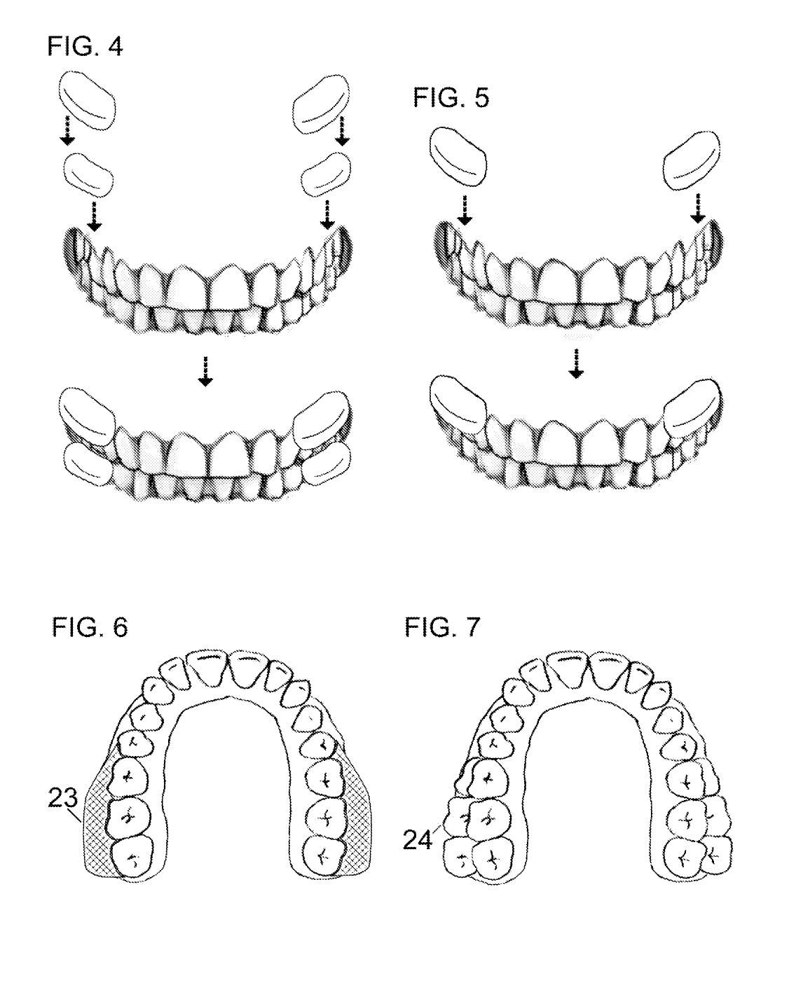

There can be one or multiple cheek pads. For instance, if an individual has an asymmetrical face due to an injury or deformity on one side of their face, only one cheek pad can be used in one quadrant to restore the needed cheek volume. Alternatively, multiple cheek pads can be attached to the individual's hind teeth. For instance, in one embodiment, the individual can have four cheek pads (FIG. 4), each of which is attached to its corresponding quadrant's hind teeth. Alternatively, the individual can have two cheek pads (FIG. 5), each attached either to its corresponding maxilla or mandible quadrant's hind teeth.

Arriving at the exact shape of each cheek pad and manufacturing it can be achieved a number of different ways and will be discussed in further detail later in this patent. So long as each resulting cheek pad's shape will meet the purpose of this invention and the requirements outlined in the above two paragraphs, any currently known or future means of making the shape of each cheek pad should be considered covered under this patent.

Attaching the cheek pads to the hind teeth can be achieved through many means of attachment. Each cheek pad can be attached either through a permanent, partially removable, or completely removable means of attachment to an individual's hind teeth. In this invention, one or multiple teeth can be used to hold each cheek pad. The selection of which and how many teeth to use to support the cheek pad will depend on the means of attachment being used, the individual's needs, dental condition, etc.

### DETAILED DESCRIPTION OF EMBODIMENTS

Embodiments for Making the Cheek Pad Without a Dental Impression

In these embodiments, a dental professional custom makes each individual's cheek pad by molding a pliable substance that will later harden (e.g. Ethylene-vinyl acetate, dental alginate, etc.). For the purpose of this entire invention, a dental professional is defined as at least one person who knows enough about the dental arts to competently perform any of the tasks involved in the making, attachment, or removal of any component of a cheek plumping apparatus. Such dental professionals include, but are not limited to dentists, orthodontists, dental laboratory technicians, dental auxiliary, or any group of people or a company involved in the manufacturing of any components of the cheek plumping apparatus, its attachment, or its removal.

During a process of molding and fitting in the individual's mouth, the dental professional can massage and squeeze the individual's cheek to cause the pliable substance to take on the thickness and shape that achieves the most desirable and natural appearance in the cheek. The pliable substance can be further molded after being removed from the individual's mouth. This pliable substance, once hardened, can then either become the actual cheek pad that will then be attached to the individual's teeth, or it can be used to make a mold; this mold then being used to create the actual cheek pad. It will likely be necessary to further trim these molded cheek pads either for comfort, to make the pad look more like a continuation of the individual's natural teeth, to prepare the side of the cheek pad facing the teeth for the means of attachment to the hind teeth, etc. For instance, the cheek pad can be fitted with the rod that will be used to attach the cheek pad to a headgear tube on a molar band on one of the individual's hind teeth. The cheek pad can optionally be coated with a substance to make it biocompatible and least likely to irritate any surface of the mouth and cheeks that it touches.

### Embodiment for Making the Cheek Pad Via Impression and Model of Teeth

In an alternative embodiment, a dental professional custom makes an individual's cheek pad by making at least one impression and at least one model of an individual's teeth, similar to techniques used to make dentures and orthodontic retainers, and building the cheek pad upon and with the assistance of that model of the individual's teeth. Thus, for instance, if the cheek plumping apparatus is being made by attaching cheek pads to a vacuum formed orthodontic retainer, after the polypropylene has been vacuum formed onto the dental model, a pliable acrylic cheek pad can then be molded and bonded onto the retainer (FIG. 6, 23). Alternatively, trimmed denture teeth can be bonded to any such removable dental appliance after it is made on a dental model (FIG. 7, 24).

### Embodiment for Pre-Manufactured Cheek Pads

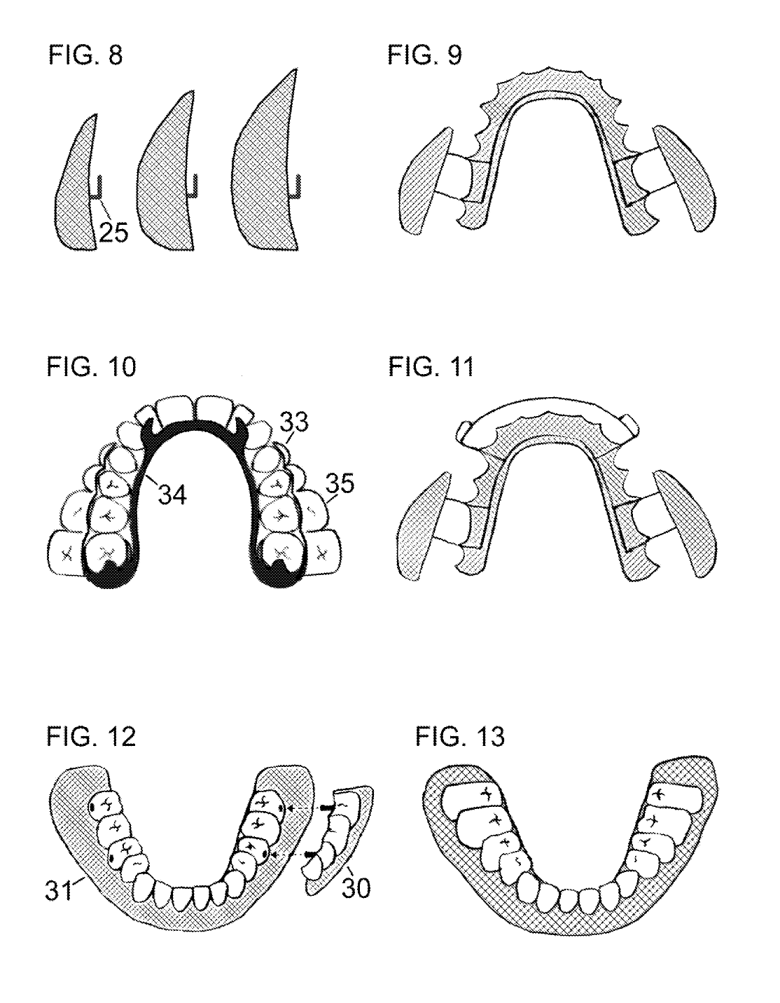

In yet a different embodiment, the cheek pad is pre-manufactured in one or multiple sizes (FIG. 8) and then provided to the dental professional. As described earlier, many different shapes, sizes, and thicknesses of cheek pads can be made so long as they meet the requirements and limitations discussed earlier. Such pre-manufactured cheek pads can optionally have incorporated in them the means for attachment to an individual's teeth. For instance, they can be pre-manufactured with the rod (FIG. 8, 25) which will be used to attach to a headgear tube on a molar band. The dental professional attaching these pre-manufactured cheek pads to the individual's hind teeth may also have the option of further trimming and shaping these prefabs for a more customized fit.

### Permanent Cheek Pad Attachment Embodiment

In the permanent cheek plumping apparatus embodiment, at least one cheek pad is attached to the hind teeth such that the individual is not able to readily remove them, and will need the care of a dental professional to remove them. Many permanent means of attachment exist, and will likely be developed in the future, but so long as they meet the criteria of the cheek pads not being readily removable by the individual, they are to be considered part of this invention. For instance, some permanent means for attaching the cheek pads to the teeth include, but are not limited to the use of dental cement or dental glue, screws, nails, or the use of molar bands or other tooth bands (FIG. 1, 15) (cemented or otherwise firmly attached to at least one hind tooth), or the combination of such techniques. The cheek plumping apparatus can also be attached permanent to at least one dental restoration. For instance, cheek pads can be attached to dental implants, crowns, or bridges. When a permanent means of attachment pertains to attaching a cheek pad to anything other than natural teeth, its definition also encompasses incorporating the cheek pad into the non-natural tooth object. Thus, for instance, if a cheek pad is desired to exist on a molar tooth implant, the molar tooth implant's crown itself can be made thicker in the direction of the cheek to achieve this goal.

### Partially Removable Cheek Plumping Apparatus Embodiments

In the partially removable cheek plumping apparatus embodiment, a combination permanent and removable means of attaching the cheek pad to at least one of the individual's hind teeth are used. Here, the mounting, upon which the pad will be attached, will have a permanent means of attachment to at least one of the individual's hind teeth, while the cheek pad itself will be removable from said mounting. The mounting is defined as any base, backing, or setting designed to receive and attach to the cheek pad via a removable means of attachment. Some examples of a mounting include, but are not limited to, a headgear tube on a molar band, or a nut or any other similar hole attached to, or incorporated into an individual's hind tooth.

The permanent means for attaching the mounting is such that the individual will not be able to remove the mounting on a day to day basis and will need assistance from a dental professional to do so. For instance, some permanent means for attaching the mounting to the teeth include, but are not limited to the use of dental cement or dental glue, screws, nails, or the combination of such techniques.

The removable means of attaching the cheek pad to the mounting includes any detachable means. Such removable means of attachment include, but are not limited to, using a system of at least one slide rail, snap fastener, bolt, clip, or magnet to attach the cheek pad to the mounting. Thus, part of the removable means of attachment is incorporated into the cheek pad. In this way, the individual is able to remove the cheek pad itself, while leaving the mounting behind.

For instance, for each quadrant, the inner face of a cheek pad (FIG. 2, 26) can be permanently attached to a protruding rod (FIG. 2, 27) that the individual can then insert into a headgear tube (FIG. 2, 28) on a molar band (FIG. 2, 29) that is cemented to one of the hind teeth. In this partially removable cheek plumping apparatus, the removable cheek pad must attach to the base securely enough such that it will not dislodge or otherwise move around in the individual's mouth until the individual wishes to remove it. It is preferable to have an attachment mechanism that snaps, clips or locks into place, as an extra measure for immobility.

In an alternative partially removable cheek plumping apparatus embodiment, the male component of a snap can be permanently attached to the cheek pad. Then, if at least one of the corresponding hind teeth is a dental restoration, the female component of the snap can be incorporated into the outer face of that dental restoration. A dental restoration in this invention is defined as a permanent replacement of at least a significant part of at least one tooth. Examples of dental restoration include, but are not limited to implants, crowns, and bridges. Thus, the individual will be able to simply snap the cheek pad into place, and remove it whenever they wish.

### Completely Removable Cheek Plumping Apparatus Embodiments

In the completely removable cheek plumping apparatus embodiment, the means for attachment is such that the cheek plumping apparatus is completely removable from the mouth. Thus, the individual wearing the cheek plumping apparatus can remove the entire cheek plumping apparatus whenever they wish. The cheek plumping apparatus must have a means for attachment to at least one tooth securely enough such that the cheek plumping apparatus will be held in place and will not dislodge or otherwise move around in the individual's mouth until the individual wishes to remove it.

Such removable means of attachment include, but are not limited to, using straps, bands (FIG. 1, 15), hooks or clips around at least one tooth to attach at least one cheek pad to at least one tooth.

Removable means of attachment also include attaching or incorporating at least one cheek pad into at least one removable dental appliance like structures that will serve to hold the cheek pad on the teeth securely. A removable dental appliance is defined in this invention as any device used to align, repair, protect, or replace teeth that an individual can take out of their mouth. However, for the purpose of this invention, the removable dental appliance like structures is defined as an appliance that will be made like and look like any removable dental appliance, but will only serve the purpose of holding at least one cheek pad in place on the outer face of at least one hind tooth.

Examples of such removable dental appliance like means of attachment include, but are not limited to any removable dental appliances that is made like, or resembles orthodontic retainers like Hawley retainers (FIG. 9), clear vacuum formed retainers (FIG. 7), or pressure forming retainer, partial denture like structures (FIG. 10) (without simulation teeth), mouth guards, or splints that are custom made for each individual. Depending on the needs of the individual, there can be either a maxilla cheek plumping apparatus with such a removable dental appliance means of attachment, mandible one, or both. Thus the purpose of these removable dental appliance structures is to removably attach at least one cheek pad securely on the hind teeth in the individual's mouth.

### Cheek Pads Attached to Functional Removable Dental Appliance Embodiments

An alternative embodiment of this invention entails incorporating or attaching at least one cheek pad to at least one removable dental appliance that is serving a purpose other than just holding the cheek pad. In other words, where in the previous embodiment, the removable dental appliance like structure was simply a part of the cheek plumping apparatus, and only served to attach and hold the cheek pads on the teeth, in this embodiment, the removable dental appliance has a functional purpose in and of itself. Any removable dental appliance can be used for this embodiment, and should be considered as part of this invention's means for attachment, so long as it is capable of being attached to and holding at least one cheek pad. Such removable dental appliances include, but are not limited to orthodontic retainers such as Hawley retainers, clear vacuum formed retainers, or pressure forming retainer, dentures, clear aligners, mouth guards, or splints that are custom made for each individual. For the purpose of this invention, a denture can be either a partial denture, or a full denture. Depending on the needs of the individual, there can be either cheek pad(s) attached or incorporated into a maxilla removable dental appliance, a mandible removable dental appliance, or both.

For instance, in one embodiment, at least one cheek pad can be attached or incorporated into at least one orthodontic retainer (FIG. 11) that is being used to align the individual's teeth. Or, in a different embodiment, at least one cheek pad can be attached or incorporated into at least one denture (FIG. 12) that is serving to replace at least one missing tooth.

In some embodiments, the cheek pad is permanently attached to, or incorporated into the removable dental appliance, while in other embodiments, the cheek pad uses a removable means of attachment to the removable dental appliance. For instance, in a permanent attachment embodiment, at least one cheek pad is permanently attached or incorporated into a denture (FIG. 13), such that the individual wearing the denture cannot remove the cheek pad from the denture without the assistance of a dental professional.

In the removable means of attachment embodiment, at least one cheek pad is attached to at least one removable dental appliance using a means such that the cheek pad (FIG. 12, 30) can be removed by the individual, leaving behind a non-cheek plumping removable dental appliance (FIG. 12, 31). The cheek pads can be held in place upon the removable dental appliance using any removable technique that holds them securely and prevents them from dislodging or otherwise moving around in the individual's mouth. Such attachment techniques include but not limited to slide rails, snaps, clips, or magnets.

### Incorporating a Holding Arch into the Cheek Plumping Apparatus Embodiment

In this embodiment, an additional component to the cheek plumping apparatus comprises incorporating into the cheek plumping apparatus at least one holding arch (FIG. 2, 32) that prevents the pressure of the individual's cheeks (FIG. 2, 17) that are pushing on the cheek pads from moving any of the individual's teeth over time. A holding arch is defined in this invention as a relatively rigid structure of any material which roughly goes around the dental arch or across the floor or roof of the mouth from the left quadrant of the hind teeth to the right quadrant of the hind teeth, and attaches at each end to the cheek plumping apparatus in that quadrant. Such a movement prevention holding arch is an important component of any cheek plumping apparatus embodiment that does not go entirely around the arch of the individual's teeth, or if it does go entirely around, is soft and therefore does not provide for sufficient structural support in order to prevent any of the hind teeth from moving in a mesial direction due to the pressure of the cheeks upon the cheek pads.

### Embodiments of Means to Minimize the Visibility of Cheek Plumping Apparatus

In many people, when they smile or significantly open their mouth, not only do their front teeth become visible, but their hind teeth also become visible. Thus, for such individuals, it is greatly preferable that at least a part, if not all, of the cheek plumping apparatus should be camouflaged, in order to minimize or prevent it from being noticed. This can be accomplished in a number of ways.

The visibility of any component of the cheek plumping apparatus can be minimized by making it be a similar color as the natural tissue in that component's corresponding region of the mouth's interior. For instance, where a region of the cheek plumping apparatus lies upon the teeth, that region is to be made the same color as the teeth. Or where a region of the cheek plumping apparatus lies upon the gums, that region is to be made the same color as the gums.

Another method of reducing the visibility of any component of the cheek plumping apparatus entails making that component from a transparent material. For instance, a clear vacuum formed retainer is a transparent means of attachment (FIG. 7), and when worn, is hart to see. In addition, if the cheek pads themselves are made of a transparent clear material, they will also be difficult to notice.

In addition, any component of the cheek plumping apparatus which lies over the individual's teeth can be camouflaged by making it look like a continuation of the individual's teeth. For instance any cheek pad will become less noticeable if it is given the same shape, color and general appearance as the individual's teeth (FIG. 7, 24, FIG. 10, 35). The cheek pad can also be made to look like the teeth and gums as well (FIG. 12, 30), if the gums are also visible upon smiling. This can, for instance, be accomplished by making the outer face of the cheek pad from trimmed denture teeth, or by sculpting the cheek pad's acrylic to look like teeth and gums. It is preferable that the teeth like cheek pads should be contoured to taper down in thickness as they approach the first bicuspid's region (FIG. 10, 33), such that, if they come into view, they appear to be a continuation of the individual's hind teeth.

In addition, any component of the cheek plumping apparatus' means of attachment can be made less visible by making it such that as little of the means of attachment extends into the outer face of the teeth as possible. Thus, for instance, if a partial denture like means of attachment is being used (FIG. 10, 34), as little of the denture will extend into the outer face of the teeth as possible, particularly with regard to the front teeth.

A combination of any of the above techniques can also be used to minimize the visibility of the cheek plumping apparatus.

### BEST MODE FOR CARRYING OUT THE INVENTION

In the preferred embodiment of this invention, teeth like cheek pads are attached to each quadrant on a maxilla vacuum formed retainers and mandible vacuum formed retainers (FIG. 7). First, a dental professional takes impressions and make a model of both hard and soft tissues in both the maxilla and mandible sides of an individual's teeth. Then, the dental professional vacuum forms a thin sheet of polypropylene onto the dental models. The vacuum formed retainers are trimmed to include some coverage of the hind teeth. Next, the dental professional uses pliable wax to form temporary cheek pads inside the individual's mouth. During such a custom fitting stage, the size, location and shape of the cheek pads are experimented with, and the four shapes and locations which yield the most attractive and comfortable results are decided upon.

After carefully removing the wax cheek pads, the dental professional uses their shape, size, and location as guides to form and bond permanent acrylic cheek pads onto the vacuum formed retainers. These acrylic cheek pads are made to match the color of the individual's hind teeth, and are sculpted and carved to look like continuations of the individual's hind teeth (FIG. 7, 24). Upon completion, the new cheek plumping apparatuses go for a final fitting, and additional adjustments are made if necessary.

What is claimed is:

1. A cheek plumping apparatus which causes at least one of an individual's cheeks to be pushed outwards:
comprising at least one cheek pad and at least one biocompatible means of attaching said cheek pad to the exterior of at least one of the individual's hind teeth;
wherein the means of attachment of the cheek pad must be such that the cheek pad stays securely in place, and does not dislodge or otherwise move around in the individual's mouth;
wherein each cheek pad must be biocompatible and made of at least one durable material such that it will not tear or break apart while in use, and must be made of a material that is safe to use in a human mouth, and its surface is such that it will not irritate or otherwise disturb the mouth's tissues or compromise the health of the individual wearing it when worn for hours at a time;
and wherein each cheek pad must have a size and shape and thickness such that it will not irritate any of the mouth's tissues, such that the pad cannot be so large as to push on or irritate the mucobuccal fold or the anterior folds in the cheek's cavity, and the height of the pad does not interfere with the movement of one's mouth or obstruct the individual from being able to close their mouth and jaws completely.

2. The cheek plumping apparatus of claim 1 including the further step of at least one cheek pad covering at least part of at least one hind tooth.

3. The cheek plumping apparatus of claim 1 including the further step of a dental professional custom making and custom fitting each individual's cheek pad by molding a pliable substance that will later harden and become the actual pad that will be placed upon the individual's teeth.

4. The cheek plumping apparatus of claim 1 including the further step of a dental professional custom making and custom fitting each individual's cheek pad by:
molding a pliable substance that will later harden;
then using the hardened pliable substance to make a mold;
and then using this mold to create the actual pad.

5. The cheek plumping apparatus of claim 1 including the further step of custom making each individual's cheek pad by:
making at least one impression and at least one model of an individual's teeth, similar to techniques used to make dentures and orthodontic retainers;
and building the cheek pad with the assistance of that model of the individual's teeth.

6. The cheek plumping apparatus of claim 1 including the further step of cheek pads of at least one size being pre-manufactured and then provided to dental professionals.

7. The cheek plumping apparatus of claim 1 including the further step of cheek pads of at least one size being pre-manufactured and then provided to dental professionals, wherein the pre-manufactured cheek pads have incorporated in them at least part of the means for attachment to an individual's teeth.

8. The cheek plumping apparatus of claim 1 including the further step of using a permanent means of attaching the cheek pad to at least one of the individual's hind teeth, such that the individual will not be able to remove the cheek pad on a day to day basis and will need assistance from a dental professional to do so.

9. The cheek plumping apparatus of claim 1 including the further step of using a combination permanent and removable means of attaching the cheek pad to at least one of the individual's hind teeth, such that:
the mounting (upon which the pad will be attached) will have a permanent means of attachment to at least one of the individual's hind teeth, while the cheek pad itself will be removable from said mounting, wherein:
the means for attaching the mounting is such that the individual will not be able to remove the mounting on a day to day basis and will need assistance of a dental professional to do so;
the means of attaching the cheek pad to the mounting includes any biocompatible detachable means;
and the individual would be able to remove the cheek pad itself, while leaving the mounting behind.

10. The cheek plumping apparatus of claim 1 including the further step of the cheek plumping apparatus having a means for attachment such that the cheek plumping apparatus is completely removable from the mouth wherein:
the individual wearing the cheek plumping apparatus can remove the cheek plumping apparatus whenever they wish;
and the cheek plumping apparatus has a means for attachment to at least one tooth securely enough such that the cheek plumping apparatus will be held in place and will not dislodge or otherwise move around in the individual's mouth until the individual wishes to remove it.

11. The cheek plumping apparatus of claim 1 including the further step of making the cheek plumping apparatus by attaching at least one cheek pad to at least one removable dental appliance like structures who's function is solely to hold the cheek pad on the teeth securely.

12. The cheek plumping apparatus of claim 1 including the further step of at least one cheek pad being permanently attached to at least one removable dental appliance which has a functional purpose in and of itself and other than just holding the cheek pad.

13. The cheek plumping apparatus of claim 1 including the further step of at least one cheek pad having a removable means for attachment to at least one removable dental appliance which has a functional purpose in and of itself and other than just holding the cheek pad wherein:
each cheek pad can be removed and detached from the removable dental appliance by the individual, leaving behind a non-cheek plumping removable dental appliance;
and each cheek pad is held in place upon the removable dental appliance using any removable means of attachment that holds it securely and prevents it from dislodging or otherwise moving around in the individual's mouth.

14. The cheek plumping apparatus of claim 1 including the further step of using a permanent means of attachment to attach at least one cheek pad to at least one dental restoration.

15. The cheek plumping apparatus of claim 1 including the further step of incorporating into the cheek plumping apparatus a holding arch that prevents the pressure of the individual's cheeks that are pushing on the cheek pads from moving any of the individual's teeth over time.

16. The cheek plumping apparatus of claim 1 including the further step of minimizing at least one component of the cheek plumping apparatus from being seen by having it be of a similar color as the natural tissues in that component's corresponding region of the mouth's interior.

17. The cheek plumping apparatus of claim 1 including the further step of minimizing at least one component of the cheek plumping apparatus from being seen by having it made from a material which is in at least one region transparent.

18. The cheek plumping apparatus of claim 1 including the further step of minimizing at least one component of the cheek plumping apparatus from being seen by making it look like at least one tooth.

19. The cheek plumping apparatus of claim 1 including the further step of minimizing at least one component of the cheek plumping apparatus' means of attachment from being seen by making it such that as little of the means of attachment will extend into the outer face of the teeth as possible.
